(19) United States
(12) Patent Application Publication
(10) Pub. No.: US 2026/0200856 A1
(43) Pub. Date: Jul. 16, 2026

(54) PROCESS FOR THE PREPARATION OF ONE OR MORE COMPOUNDS OF THIOPYRIDINONE TYPE

(71) Applicant: L'Oreal, Paris (FR)

(72) Inventors: Ozkan Sari, LE THILLAY (FR); Jinzhu Xu, AULNAY-SOUS-BOIS (FR); Patricio Guerreiro, AULNAY-SOUS-BOIS (FR)

(73) Assignee: L'Oreal, Paris (FR)

(21) Appl. No.: 19/133,985

(22) PCT Filed: Nov. 29, 2023
(86) PCT No.: PCT/EP2023/083491

§ 371 Date: May 29, 2025

(30) Foreign Application Priority Data
Nov. 30, 2022 (FR) FR2212589

(51) Int. Cl. C07D 213/82; A61K 8/49; A61Q 19/02

(52) U.S. Cl. CPC C07D 213/82; A61K 8/4933; A61Q 19/02; A61K 2800/10

(57) ABSTRACT

Process for the preparation of one or more compounds of thiopyridinone type The present invention relates to a process for the preparation of one or more compounds of thiopyridinone type of formula (I) and their optical isomers, geometrical isomers and tautomers, and also their organic or inorganic acid or base salts, and their solvates, such as hydrates. The invention also relates to one or more compounds of thiopyridinone type of formula (I″) and a composition employing said compounds.

The present invention relates to a process for the preparation of one or more compounds of thiopyridinone type of formula (I), as described below, and their optical isomers, geometrical isomers and tautomers, and also their organic or inorganic acid or base salts, and their solvates, such as hydrates.

The invention also relates to one or more compounds of thiopyridinone type of formula (I″).

N-[(2-Thioxo-1,2-dihydropyridin-3-yl)carbonyl]glycine, belonging to the family of the thiopyridinones, is a compound, the properties of which are generally advantageous in the field of cosmetics, in particular in order to be used as whitening, lightening and/or depigmenting agent for keratin materials, in particular the skin.

N-[(2-Thioxo-1,2-dihydropyridin-3-yl)carbonyl]glycine can, for example, be prepared by means of a synthesis route described in the scientific paper entitled “Synthesis of N-(2-mercaptopyridyl-3-formyl)-N-alkyl glycine and the corresponding disulfides” (Luo, Y. L., Yang, Z. X. and Peng, S. X., Div. Med. Chem., China Pharm. Univ., Nanjing, 21009, Peop. Rep. China, Yaoxue Xuebao, 25(5), 374-8 (1990)).

More generally, the compounds of thiopyridinone type corresponding to the following formula (A):

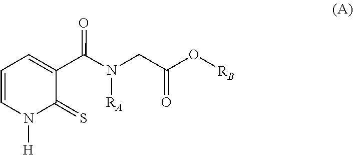

in which formula (A):
   R_{A} represents a hydrogen atom or a saturated linear C₁-C₆ alkyl group,
R_{B} represents a hydrogen atom, a saturated linear C₁-C₁₀ alkyl group, a saturated branched C₃-C₁₀ alkyl group or a phenyl(C₁-C₆)alkyl group;
are capable of being prepared according to the following synthesis route:

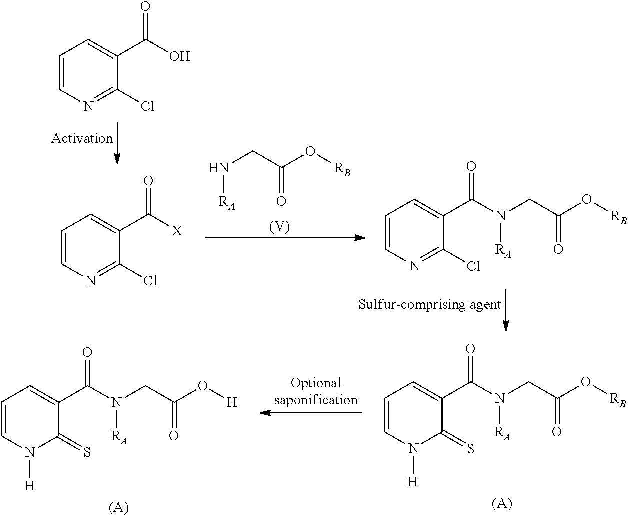

More particularly, in accordance with such a reaction scheme, the process for the preparation of the compounds of thiopyridinone type of formula (A) comprises at least the following successive stages:
a) at least a stage of activation of the carboxylic acid group of 2-chloronicotinic acid of following formula:

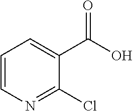

in the presence of at least one agent for activation of carboxylic acids according to conventional methods of activation of acids (described, for example, in Comprehensive Organic Transformations by R. Larock, published by Wiley VCH, in the chapter Interconversion of Nitriles, Carboxylic Acids and Derivatives, 1989) to result in a compound corresponding to the following formula (W):

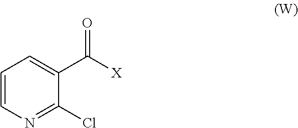

in which formula (W):
   X forms an acid halide, and a mixed anhydride,
b) at least a stage of reaction of the compound of formula (W) with an amine of following formula (V):

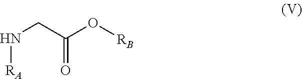

to result in a compound of following formula (Y):

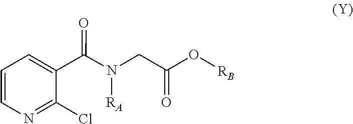

in which formulae (V) and (Y), R_{A} and R_{B} have the same meanings as in the formula (A); then
c) at least a stage of exchange between the chlorine atom and the sulfur atom using reagents such as sodium disulfite, thiourea, sodium thiosulfate or thioacetic acid in basic medium in order to result in one or more compounds of formula (A):

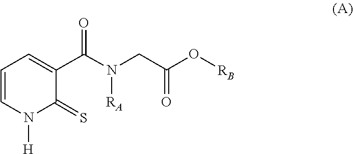

d) and optionally, for the compounds of formula (A) for which the radical R_{B} represents a hydrogen atom (i.e., comprising a carboxylic acid group), the latter can also be obtained by virtue of the implementation of at least one additional stage of saponification of the corresponding esters using one or more inorganic bases, for example with sodium hydroxide (NaOH) or lithium hydroxide (LiOH), followed by an acidification.

It results from this that the non-esterified thiopyridinone compounds, that is to say those corresponding to the formula (A) in which R_{B} represents a radical other than the alkyl or phenylalkyl groups, can be prepared from such a process, either by employing one or more additional saponification stages (optional stage d)) or directly from an amine of corresponding formula (V) (stage b)).

However, such a process still exhibits a certain number of disadvantages (for example, additional saponification stage, impurity), in particular when the desired final thiopyridinone compounds are not esterified.

On the one hand, when the thiopyridinone compound of formula (A), in which the radical R_{B} corresponds to a hydrogen atom, is obtained directly from an amine of corresponding formula (V), that is to say without employing one or more additional saponification stages, the process thus implemented still too often poses difficulties of feasibility on the industrial scale and requires better optimization.

On the other hand, when the thiopyridinone compound of formula (A), in which the radical R_{B} corresponds to a hydrogen atom, is obtained from the implementation of one or more additional saponification stages, then the process exhibits in particular the disadvantages of enhancing the energy consumption, of increasing the amount of reagents used and of generating additional effluents. In addition, such additional stages can cause a significant drop in the yield.

It results from this that the process for the preparation of non-esterified thiopyridinone compounds of formula (A) does not result in entirely satisfactory yields and its implementation necessitates being further optimized, in particular in terms of number of stages and/or of quality of the product at the industrial scale.

In the light of the above, there thus exists a real need to provide a novel process for the preparation of compounds of thiopyridinone type corresponding to the formula (I), as described below, which does not exhibit the abovementioned disadvantages.

In other words, one of the aims of the present invention is to provide a process for the preparation of compounds of thiopyridinone type having in particular a better yield and the implementation of which is more optimized, specifically on the industrial level. In addition, the compounds of formula (I) with the process of the invention exhibit a high purity greater than that obtained by the saponification of the ester.

A subject-matter of the present invention is thus in particular a process for the preparation of at least one compound of following formula (I):

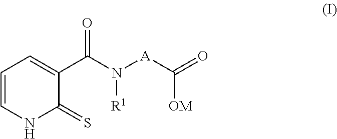

in which formula (I):
A represents a saturated or unsaturated, preferably saturated, cyclic or acyclic, preferably acyclic, linear or branched, divalent hydrocarbon chain comprising from 1 to 10 carbon atoms, optionally interrupted by one or more heteroatoms or groups, such as an oxygen atom, a sulfur atom, an NR₂ or (thio)carbonyl group, or their combinations, such as thioester, with R₂ representing a hydrogen atom or a C₁-C₄ alkyl group;
M represents a hydrogen atom, an alkali metal or alkaline earth metal, an ammonium group;
R¹ represents a hydrogen atom, a linear or branched C₁-C₁₀ alkyl group, optionally substituted by an OR₃, SR₃ group with R₃ corresponding to a hydrogen atom or a linear or branched C₁-C₁₀ alkyl group,
and also one of its optical isomers, geometrical isomers and tautomers, and also one of its organic or inorganic acid or base salts, and one of its solvates, such as hydrates;
   said process taking place according to the following synthesis scheme (1):

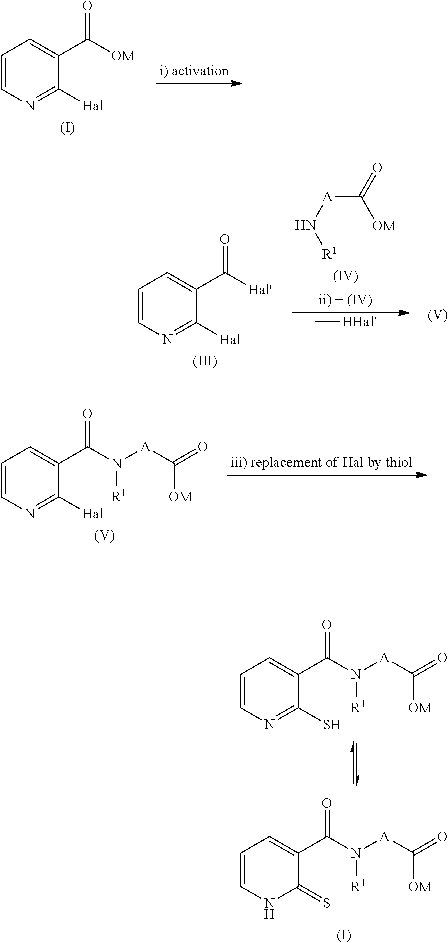

in which:
   Hal and Hal′, which are identical or different, represent a halogen atom, and
   M, A and R¹ have the same meanings as in the formula (I);
at least stage (iii) is at acidic pH, i.e. less than 7.

The process according to the invention thus makes it possible to achieve the objectives as described above, that is to say that it results in a satisfactory yield and exhibits an optimized implementation on the industrial level, in particular with respect to the known processes for obtaining thiopyridinone compounds. In addition, the compounds are extremely pure (greater than or equal to 99%, indeed even greater than or equal to 99.0% by HPLC).

Advantageously, the process according to the invention makes it possible to reduce the amounts of reagents used and also the effluents generated, and its implementation can result in a saving in energy, in particular compared with the known processes for obtaining thiopyridinone compounds employing one or more additional saponification stages.

Another subject-matter of the present invention is a compound of following formula (I″):

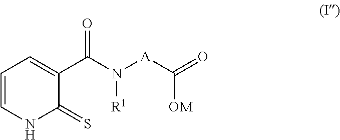

in which formula (I″):
   M, A and R¹ have the same meanings as in the formula (I),
and also their optical isomers, geometrical isomers and tautomers, and also their organic or inorganic acid or base salts, and their solvates, such as hydrates,
with the exception of the following compounds (X) and (XI) and their tautomers (X′) and (XI)′:

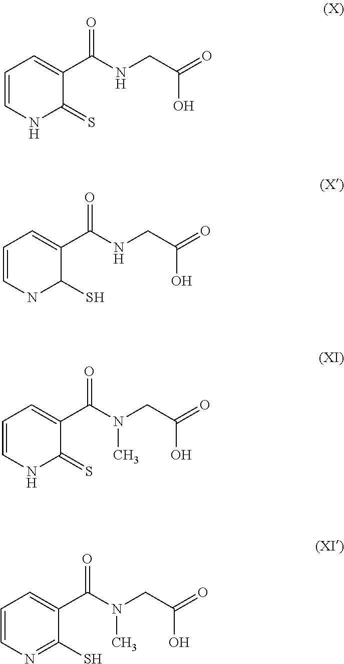

Furthermore, the present invention also relates to a composition comprising, in a physiologically acceptable medium, at least one compound of formula (I″), its salts and/or isomers and/or solvates, as described above.

Other subject-matters, characteristics, aspects and advantages of the invention will become even more clearly apparent on reading the description and the example which follow.

In that which will follow, and unless otherwise indicated, the limits of a range of values are included in this range, in particular in the expressions “of between” and “ranging from . . . to . . . ”.

Moreover, the expression “at least one” used in the present description is equivalent to the expression “one or more”.

In addition, the expression “at least” used in the present description is equivalent to the expression “greater than or equal to”. Finally, in a way known per se, the term “Cₙ” compound or group denotes a compound or a group containing, in its chemical structure, “n” carbon atoms.

The HPLC purity corresponds to the relative purity expressed as percentage of area, measured at the maximum absorption wavelength (λₘₐₓ) of the product analysed.

Depending on the requirements of the stages of the process of the invention, the reaction media can be maintained as an acidic or basic medium.

An acidic pH, i.e. of less than 7, can be adjusted by means of the addition of an acidifying agent, optionally in aqueous solution, the acidifying agent being organic or inorganic, in particular inorganic. Mention may be made, among the acidifying agents, by way of example, of inorganic or organic acids, such as hydrochloric acid, orthophosphoric acid, sulfuric acid, carboxylic acids, such as acetic acid, tartaric acid, citric acid or lactic acid, and sulfonic acids.

Preferably, the acidifying agent is an inorganic acid, in particular hydrochloric acid.

Preferably, the acidifying agent is introduced in aqueous solution.

A basic pH, i.e. of greater than 7, can be adjusted by means of the addition of an alkaline agent, optionally in aqueous solution, said alkaline or basifying agent being organic or inorganic, preferably inorganic.

The alkaline agent(s) can be chosen from inorganic, organic or hybrid alkaline agents.

Within the meaning of the present invention, the terms “alkaline agent” and “basifying agent” are used without distinction.

The basifying agent(s) can be inorganic alkaline agents, preferably chosen from the group consisting of alkali metal hydroxides or alkaline earth metal hydroxides, such as lithium hydroxide, sodium hydroxide, potassium hydroxide, alkali metal or alkaline earth metal (bi)carbonates, such as sodium or potassium (bi)carbonates, and their mixtures. The basifying agent(s) can be organic alkaline agents, preferably chosen from the group consisting of mono(C₁-C₆)(hydroxy)alkylamines, di(C₁-C₆)(hydroxy)alkylamines, tri(C₁-C₆)(hydroxy)alkylamines (preferably tri(C₁-C₆)(hydroxy)alkylamines), saturated or unsaturated cyclic amines which are aromatic, such as pyridine, or non-aromatic, optionally substituted by one or more (C₁-C₄)alkyl groups, such as tetrahydropyridine optionally substituted by one or more (C₁-C₄)alkyl groups, piperidine optionally substituted by one or more (C₁-C₄)alkyl groups or piperazine optionally substituted by one or more (C₁-C₄)alkyl groups. Preferably, the alkaline agents of the invention are tertiary amines.

Preferably, the basifying agents are chosen from the group consisting of alkali metal or alkaline earth metal hydroxides, in particular sodium hydroxide, alkali metal or alkaline earth metal (bi)carbonates, in particular sodium or potassium (bi)carbonates, and tri(C₁-C₆)(hydroxy)alkylamines, in particular tri(C₁-C₆)alkylamines, especially triethylamine.

More preferentially, the basifying agents are inorganic and are chosen from the group consisting of alkali metal or alkaline earth metal hydroxides, alkali metal or alkaline earth metal (bi)carbonates, and their mixtures, in particular alkali metal or alkaline earth metal hydroxides, in particular sodium hydroxide.

Preferably, the organic solvent(s) used in the process of the invention is (are) non-polar aprotic solvent(s), i.e. has (have) a dielectric constant E ranging from 1 to 11 and a dipole moment p ranging from 0 to 2, in particular chosen from the group consisting of n-hexane, cyclohexane, 1,4-dioxane, carbon tetrachloride (CCl₄), benzene, tetrachloroethylene (Cl₂C═CCl₂), toluene, carbon disulfide (CS₂), trichloroethylene (Cl₂C═CHCl), diethyl ether (Et₂O), trichloromethane (CHCl₃), bromobenzene (PhBr), chlorobenzene (PhCl), ethyl acetate (CH₃C(O)OEt or AcOEt), dimethyl ether (DME), tetrahydrofuran (THF), methyltetrahydrofuran (2-MeTHF), dichloromethane (CH₂Cl₂), dichloroethane (ClCH₂CH₂Cl) and 1,1-dichloroethane (Cl₂CHCH₃). Preferably, the organic solvent(s) exhibit a dielectric constant E ranging from 1 to 7 and a dipole moment μ ranging from 0 to 2. More preferentially, the organic solvent or solvents are chosen from ethyl acetate, methyltetrahydrofuran and toluene, more preferentially ethyl acetate.

### Preparation Process

As indicated above, the process according to the invention takes place according to the reaction scheme (1), as described above, and at least stage (iii), that is to say the stage of reaction of the compound of formula (V) resulting in the compound of formula (I), is carried out at a pH strictly of less than 7.

The pH of the reaction medium is at a value strictly of less than 7 during the stage of reaction of the compound of formula (V) resulting in the compound of formula (I) being obtained (stage (iii)).

Preferably, the reaction medium during stage (iii) is at a pH of less than or equal to 6, more preferentially of less than or equal to 5, more preferentially still of less than or equal to 4.

Preferably, the reaction medium during stage (iii) is at a pH which varies from 1 to 4, in particular ranging from 2 to 3.

According to a particular embodiment of the invention, the pH is of between 6 and 2, more particularly between 5 and 3. The process according to the invention comprises preferentially successively at least the following stages:
at least a stage (i) of preparation of the compound of following formula (III):

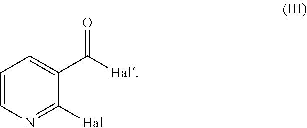

in which formula (III):
   Hal and Hal′ have the same meanings as those described above; preferably, Hal and Hal′ are identical and more preferentially represent a chlorine atom,
by activation of the carboxylic group of the compound of following formula (II):

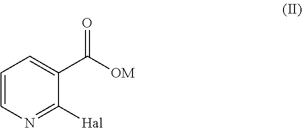

in the presence of one or more agents for activation of carboxylic acids according to conventional methods of activation of acid functions, in particular using thionyl halide, such as thionyl chloride;
in particular, the agent(s) for activation of carboxylic acids are as defined below “the agent for activation of carboxylic acids”, chosen in particular from thionyl halides, such as thionyl chloride, which is added dropwise (in particular in an amount of activating agent in excess with respect to the carboxylic acid to be activated, preferably of between 1 and 2 molar equivalents, such as 1.1 molar equivalents, with respect to (II)) onto the compound of formula (II); preferably, the compound (II) is in a non-polar aprotic solvent as defined above, in particular a solvent which is an ester of (C₁-C₆)alkyl acid and of (C₁-C₆)alkanol, such as ethyl acetate, or an aromatic solvent, such as toluene;
in particular, the reaction of stage (i) is carried out under an inert atmosphere of argon or nitrogen (at atmospheric pressure), at a temperature of between 25° C. and solvent reflux, preferably between 40° C. and 120° C., such as 77° C. or 110.6° C.,
in particular, the reaction mixture of stage (i) is left, preferably at solvent reflux, under mechanical stirring for a period of time of between 30 minutes and 10 hours, more particularly between 1 hour and 6 hours, such as 4 hours;
preferably, subsequently, the reaction mixture of stage (i) is cooled down to ambient temperature (25° C.) and preferably under an inert atmosphere;
at least a stage (ii) of reaction of the compound of formula (III) with an amine of following formula (IV):

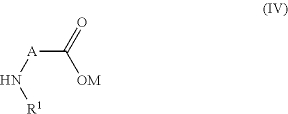

in order to obtain the compound of following formula (V):

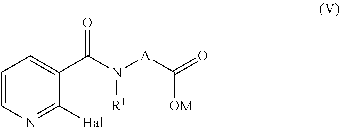

in which formulae (IV) and (V):
   A, M and R¹ have the same meanings as those indicated above for the compound of formula (I);
   in particular, stage (ii) is carried out with:
   at least one equivalent of compound (IV), preferably more than one equivalent (in particular between 1.5 equivalents and 2.5 equivalents, more particularly 2 equivalents, of compound (IV)), preferably in a polar (a)protic solvent, preferably a polar protic solvent, such as water, in particular in a volume weight ratio of between 1 and 7, better still between 2 and 5, such as 3.5, with respect to the weight of the compound of formula (II); preferably, the reaction mixture is left at a temperature of between 10° C. and 30° C., such as 20° C.+/−5° C.; preferably, the reaction medium is left under an inert atmosphere; there is subsequently in particular added at least one inorganic or organic alkaline agent, preferably an inorganic alkaline agent, such as sodium hydroxide; at least one equivalent of alkaline agent is added to the reaction medium in order to arrive at a basic pH of said medium,
   at least one compound of formula (III), in particular in a non-polar aprotic organic solvent as defined above, in particular ethyl acetate or toluene, with an alkaline agent as defined above, it being possible for said alkaline agent preferably to be in a polar (a)protic solvent, such as water, more particularly an inorganic alkaline agent, such as sodium hydroxide (preferably between 1 and 3 equivalents of alkaline agent, such as 1.5 equivalents),
   preferably, the compound of formula (III) is added dropwise onto the compound of formula (IV) at a temperature of between 10° C. and 30° C., such as 20° C.+/−5° C., and preferably under an inert atmosphere and under mechanical stirring; in particular, the reaction is maintained between 1 minute and 6 hours, more particularly between 30 minutes and 2 hours, such as one hour;
   more preferentially, during the addition of the compound of formula (III), said reaction medium is maintained at a basic pH, more particularly of between 7.5 and 10;
   preferably, the mixture is left to separate by settling, particularly at a temperature of between 10° C. and 30° C., such as 20° C.+/−5° C., and the aqueous phase is collected;
   in particular, the aqueous phase is cooled to a temperature of less than 30° C., preferably of between 20° C. and 30° C., such as 25° C.+/−5° C.;
   preferably, said phase is acidified with an inorganic or organic acid, preferably an inorganic acid, such as hydrochloric acid, in particular with at least 1 equivalent of acid, preferably between 1 and 3 equivalents, such as 1.6 equivalents; the pH of said aqueous phase is preferably of between 1 and 5, in particular between 2 and 4, such as 2.5+/−0.2;
   preferably, the aqueous phase is subsequently filtered and the solid obtained is preferably washed with a polar (a)protic solvent, preferably a polar protic solvent, such as water; said solid is then optionally dried, preferably at a temperature of greater than or equal to 20° C., more preferentially between 30° C. and 60° C., such as 50° C.+/−5° C., to result in the compound of formula (V);
   the drying stage is optional; the wet product can be used directly in the following stage;
at least one stage (iii) of thionation reaction of the compound of formula (V) to result in the compound of formula (I);
   said reaction stage (iii) taking place at a pH of less than 7 (the value 7 being excluded), that is to say that said stage takes place at an acidic pH (or in an acidic reaction medium), preferably of less than or equal to 6, more preferentially of less than or equal to 5, more preferentially still of less than or equal to 4, in particular in a pH range extending from 1 to 4, preferably from 2 to 3; according to a particular embodiment of the invention, the pH of stage (iii) is of between 6 and 2, more particularly between 5 and 3;
   said thionation reaction of the compound (V) is carried out in the presence of a thionation agent, preferably with alkaline or alkaline earth agent thiosulfate, such as sodium thiosulfate, preferably in the presence of a polar (a)protic solvent, preferably a polar protic solvent, such as water;
   preferably, the reaction medium in stage iii) is maintained under an inert atmosphere, in particular at a temperature of less than or equal to the solvent reflux, preferably at a temperature of between 50° C. and 110° C., more particularly between 60° C. and 100° C., preferentially between 70° C. and 90° C., such as 80° C.+/−3° C.; preferably for between 30 minutes and 24 hours, more preferentially between 1 hour and 10 hours, better still between 4 and 8 hours, such as 6 hours; preferably, the reaction mixture is cooled to a temperature of between 10° C. and 30° C., such as 20° C.+/−5° C.;
   iii1) the reaction mixture is subsequently adjusted preferably to a pH of between 7 and 8, in particular using at least one alkaline agent as defined above, preferably an inorganic alkaline agent, such as sodium hydroxide; preferentially, the reaction mixture is subsequently left under mechanical stirring at a temperature of between 10° C. and 30° C., such as 20° C.+/−5° C., in particular for one minute to 1 hour, such as 15 minutes; preferably, the reaction mixture is filtered and the solid is washed with in particular a polar (a)protic solvent, preferably a polar protic solvent, such as water;
   iii2) the filtrate is subsequently acidified, preferably at a temperature of greater than or equal to 40° C., such as 60° C.+/−5° C., using an organic or inorganic acid, preferably an inorganic acid, such as hydrochloric acid, until a pH of between 1 and 5, preferably between 1.5 and 3, such as 2+/−0.5, is obtained; the temperature is subsequently preferably maintained at 60° C.+/−5° C. for a period of time of in particular between 1 minute and 30 minutes, such as 15 minutes;
   preferentially, the reaction mixture is subsequently left under mechanical stirring at a temperature of between 10° C. and 30° C., such as 20° C.+/−5° C., in particular for one minute to 1 hour, such as 15 minutes; preferably, the reaction mixture is filtered and the solid is washed with in particular a polar (a)protic solvent, preferably a polar protic solvent, such as water; preferably, the solid is dried at a temperature of between 20° C. and 60° C., such as 50° C., in particular under vacuum;
   iii3) the solid can be purified by dissolving it in a polar (a)protic solvent, preferably a polar protic solvent, such as water, by adjusting the pH between 7 and 8 using in particular an alkaline agent as defined above, preferably an inorganic alkaline agent, such as sodium hydroxide; the mixture is subsequently preferably maintained under mechanical stirring, in particular between 10° C. and 30° C., such as 20° C.+/−5° C., for between 1 minute and 30 minutes, such as 5 minutes; the mixture is subsequently preferably filtered and the solid washed with a polar (a)protic solvent, preferably a polar protic solvent, such as water;
   iii4) the filtrate is subsequently optionally acidified (depending on the nature of M) using an inorganic or organic acid, preferably an inorganic acid, such as hydrochloric acid, in particular at a temperature of greater than or equal to 40° C., such as 60° C.+/−5° C.; preferably, the pH is maintained between 1 and 4, more preferentially between 1.5 and 3, such as 2+/−0.5; preferably, the mixture is subsequently left at a temperature of greater than or equal to 40° C., such as 60° C.+/−5° C., for in particular between 1 and 60 minutes, such as 15 minutes; the solid is subsequently filtered off and washed with a polar (a)protic solvent, preferably a polar protic solvent, such as water, followed in particular by drying at a temperature of greater than or equal to 20° C., more particularly at a temperature of between 40° C. and 60° C., such as 50° C., preferably under vacuum.

In accordance with a general characteristic according to the invention, stage (i), that is to say the stage of preparation of the compound of formula (III) by activation of the carboxylic group of the compound of formula (II), is preferentially carried out in the presence of one or more agents for activation of carboxylic acids, in an organic solvent.

The agent(s) for activation of carboxylic acids can be chosen from the group consisting of thionyl halides, such as thionyl chloride, oxalyl halides, such as oxalyl chloride, and their mixtures, preferentially thionyl chloride (SOCl₂).

Preferably, the activating agent is thionyl chloride (SOCl₂) and the organic solvent exhibits a dielectric constant E ranging from 1 to 7 and a dipole moment p ranging from 0 to 2.

Preferentially again, the activating agent is thionyl chloride (SOCl₂) and the organic solvent is chosen from the group consisting of ethyl acetate, methyltetrahydrofuran and toluene, more preferentially ethyl acetate.

Stage (ii), that is to say the stage of reaction of the compound of formula (III) with an amine of formula (IV) to result in a compound of formula (V), is preferentially carried out by bringing a composition comprising at least the compound of formula (III) and at least one organic solvent, preferably an aprotic organic solvent as described above, into contact with an aqueous composition comprising at least the amine of formula (IV), in the presence of one or more alkaline agents.

Advantageously, stage (ii) is thus carried out in a reaction medium, the pH of which is strictly greater than 7, i.e. thus at basic pH.

In other words, the stage of reaction of the compound of formula (III) with an amine of formula (IV) to result in a compound of formula (V) takes place in a basic medium.

In this way, in accordance with the synthesis scheme (1), the halogenated derivatives of general formula HHal′ (Hal′ as defined above), in particular hydrochloric acid, generated during stage (ii) are effectively neutralized during stage (ii).

Stage (ii) is preferentially carried out in a reaction medium, the pH of which varies from 7.5 to 10.

Stage (ii) advantageously comprises the addition of a composition comprising at least the compound of formula (III) and at least one organic solvent, preferably an aprotic organic solvent as described above, to a composition comprising at least one amine of formula (IV) and one or more alkaline agents.

In this case, the alkaline agent(s) can be added to a composition comprising at least the amine of formula (IV) before carrying out stage (ii).

Alternatively, stage (ii) advantageously comprises the simultaneous addition of a composition comprising at least the compound of formula (III) and at least one organic solvent, preferably an aprotic organic solvent as described above, and of an aqueous composition comprising one or more alkaline agents to an aqueous composition comprising at least one amine of formula (IV).

The yield of the process according to the invention is thus advantageously improved when said composition comprising at least the compound of formula (III) and said aqueous composition comprising one or more alkaline agents are added simultaneously to said aqueous composition comprising at least one amine of formula (IV).

In accordance with this alternative, the aqueous composition comprising at least the amine of formula (IV) can additionally comprise one or more alkaline agents.

In this case, the alkaline agent(s) can be added to a composition comprising at least the amine of formula (IV) before carrying out stage (ii).

The content of alkaline agent can vary from 2 to 4 molar equivalents, preferably from 2.5 to 3.5 molar equivalents, with respect to the number of moles of the compound of formula (III).

The content of amine of formula (IV) can vary from 1.5 to 3 molar equivalents, preferably from 1.8 to 2.5 molar equivalents, with respect to the number of moles of the compound of formula (III).

Preferably, the organic solvent is identical to the organic solvent employed during stage i) and exhibits in particular a dielectric constant E ranging from 1 to 7 and a dipole moment p ranging from 0 to 2.

More preferentially, the organic solvent is chosen from the group consisting of ethyl acetate, methyltetrahydrofuran and toluene, more preferentially ethyl acetate.

The organic solvent can be present in a content ranging from 70% to 90% by weight, preferably in a content ranging from 75% to 85% by weight, with respect to the total weight of the composition comprising at least the compound of formula (III).

The water can be present in a content ranging from 60% to 90% by weight, preferably in a content ranging from 70% to 80% by weight, with respect to the total weight of the composition comprising at least the amine of formula (IV).

The ratio by volume of the organic solvent to the water can vary from 3 to 1, preferably is equal to 2/1.

The reaction temperature can vary from 10° C. to 45° C., preferably varies from 15° C. to 25° C.

The duration of the reaction can vary from 10 to 120 minutes, preferably varies from 30 to 60 minutes.

Once the reaction is complete, the reaction medium can be acidified with an aqueous solution of organic or inorganic acid, preferably inorganic acid.

Mention may be made, among the acidifying agents, by way of example, of inorganic or organic acids, such as hydrochloric acid, orthophosphoric acid, sulfuric acid, carboxylic acids, such as acetic acid, tartaric acid, citric acid or lactic acid, and sulfonic acids.

Preferably, the acidifying agent is an inorganic acid, in particular hydrochloric acid.

The desired product of formula (V) precipitates from the medium and is filtered off and washed with water in order to be subsequently employed in stage (iii).

As indicated above, stage iii) is a stage of reaction of the compound of formula (V) to form a compound of formula (I).

Stage iii) is an exchange reaction between the halogen atom and the thiol —SH group or the sulfur atom to form a compound of formula (I).

Preferably, stage iii) is carried out using one or more reagents, such as alkali metal (di)sulfites, in particular sodium disulfite, alkali metal thiosulfates, in particular sodium thiosulfate, thiosulfate, thiourea or thioacetic acid, and their mixtures.

More preferentially, stage iii) is carried out using alkali metal thiosulfates, in particular sodium thiosulfate.

The amount of reagent can vary from 1.2 to 3 molar equivalents, preferably from 1.4 to 2 molar equivalents, with respect to the number of moles of the compound of formula (V).

The reaction temperature can vary from 60° C. to 110° C., preferably ranging from 70° C. to 100° C., more preferentially between 75° C. and 90° C., such as 80° C.

The reaction temperature advantageously makes it possible to limit the formation of impurities while ensuring a complete reaction.

The duration of the reaction iii) can vary from 3 to 10 hours, preferably from 4 to 8 hours, such as 6 hours.

During the reaction iii), the pH of the reaction medium is at a pH of less than 7, preferably of less than or equal to 6, more preferentially of less than or equal to 5, more preferentially still of less than or equal to 4, in particular in a pH range extending from 1 to 4, preferably from 2 to 3.

According to a particular embodiment of the invention, the pH is of between 6 and 2, more particularly between 5 and 3.

The compound of formula (V) can be subsequently filtered off and washed one or more times, preferably with water.

The process according to the invention thus makes it possible to prepare one or more compounds of formula (I), and also their optical isomers, geometrical isomers and tautomers, and also their organic or inorganic acid or base salts, and their solvates, such as hydrates.

The salts of the compounds of formula (I) comprise the conventional non-toxic salts of said compounds, such as those formed from an acid or base.

Mention may in particular be made, as salts of the compounds of formula (I), of.
the salts obtained by addition of the compound of formula (I) with an inorganic base, such as sodium hydroxide, potassium hydroxide, calcium hydroxide, ammonium hydroxide, magnesium hydroxide, lithium hydroxide, and sodium, potassium or calcium carbonates or hydrogen carbonates, for example;
the salts obtained by addition of the compound of formula (I) with an organic base, such as a primary, secondary or tertiary alkylamine, for example triethylamine or butylamine. This primary, secondary or tertiary alkylamine can comprise one or more nitrogen and/or oxygen atoms and can thus comprise, for example, one or more alcohol functions; mention may in particular be made of 2-amino-2-methylpropanol, ethanolamine, triethanolamine, 2-(dimethylamino)propanol, 2-amino-2-hydroxymethyl-1,3-propanediol or 3-(dimethylamino)propylamine.

Mention may also be made of the salts of amino acids, such as, for example, lysine, arginine, guanidine, glutamic acid or aspartic acid.

Advantageously, the salts of the compounds of formula (I) can be chosen from alkali metal or alkaline earth metal salts, such as sodium, potassium, calcium or magnesium salts, or ammonium salts.

The acceptable solvates of the compounds of formula (I) comprise conventional solvates, such as those formed during the preparation of said compounds as a result of the presence of solvents.

Mention may be made, by way of example, of the solvates due to the presence of water or of linear or branched alcohols, such as ethanol or isopropanol.

The optical isomers are in particular enantiomers and diastereoisomers.

Within the meaning of the present invention, the term “tautomer” is understood to mean the following two forms:

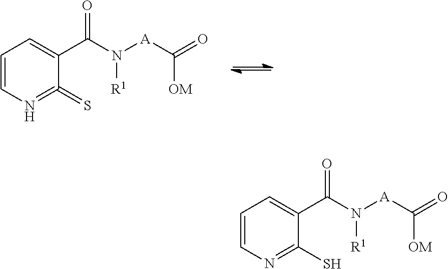

As previously indicated, A represents a saturated or unsaturated, preferably saturated, cyclic or acyclic, preferably acyclic, linear or branched, divalent hydrocarbon chain comprising from 1 to 10 carbon atoms, optionally interrupted by one or more heteroatoms or groups, such as an oxygen atom, a sulfur atom, an NR₂ or (thio)carbonyl group, or their combinations, such as thioester, with R₂ representing a hydrogen atom or a C₁-C₄ alkyl group.

In other words, A represents a saturated or unsaturated, preferably saturated, cyclic or acyclic, preferably acyclic, linear or branched, divalent hydrocarbon chain comprising from 1 to 10 carbon atoms, optionally interrupted by one or more heteroatoms, such as an oxygen atom or a sulfur atom, or groups chosen from an NR₂ or (thio)carbonyl group, or their combinations, such as thioester, with R₂ representing a hydrogen atom or a C₁-C₄ alkyl group.

In still other words, A represents a saturated or unsaturated, preferably saturated, cyclic or acyclic, preferably acyclic, linear or branched, divalent hydrocarbon chain comprising from 1 to 10 carbon atoms, which is optionally interrupted by one or more heteroatoms, such as an oxygen atom or a sulfur atom, or optionally interrupted by one or more groups chosen from an NR₂ or (thio)carbonyl group, or optionally interrupted by combinations of one or more heteroatoms and groups as previously defined, such as thioester, with R₂ representing a hydrogen atom or a C₁-C₄ alkyl group.

Preferably, in the formulae (I), (IV) and (V), A represents a saturated or unsaturated, preferably saturated, cyclic or acyclic, preferably acyclic, linear or branched, divalent hydrocarbon chain comprising from 1 to 10 carbon atoms, not interrupted by one or more heteroatoms or groups as described above.

Preferably, in the formulae (I), (IV) and (V), A represents a (C₁-C₆)alkylene group, more preferentially a (C₁-C₄)alkylene group, in particular methylene.

Preferably, in the formulae (I), (IV) and (V), R¹ represents a hydrogen atom.

In the formulae (I), (IV) and (V), when M represents an alkali metal or alkaline earth metal, M is preferably a sodium atom or a potassium atom, more preferentially sodium.

According to a preferential characteristic of the invention, in the formulae (I), (IV) and (V), R¹ represents a hydrogen atom, A represents a (C₁-C₆)alkylene group, more preferentially a (C₁-C₄)alkylene group, in particular methylene, and M represents a hydrogen atom, an alkali metal or alkaline earth metal or an ammonium group.

Advantageously, the process according to the invention makes it possible to prepare one or more compounds of formula (I) chosen from the group consisting of the compounds corresponding to the following formula (I′):

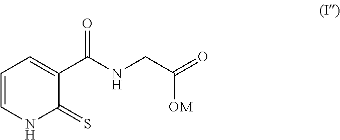

in which formula (I′):
M represents a hydrogen atom, an alkali metal or alkaline earth metal, such as a sodium atom or a potassium atom, or an ammonium group, more preferentially sodium;
and also their optical isomers, geometrical isomers and tautomers, and also their organic or inorganic acid or base salts, and their solvates, such as hydrates.

Preferably, in the formulae (I) and (I′), M represents a hydrogen atom.

The process according to the invention thus makes it possible to prepare the compound(s) of formula (I′) preferentially according to the following synthesis scheme

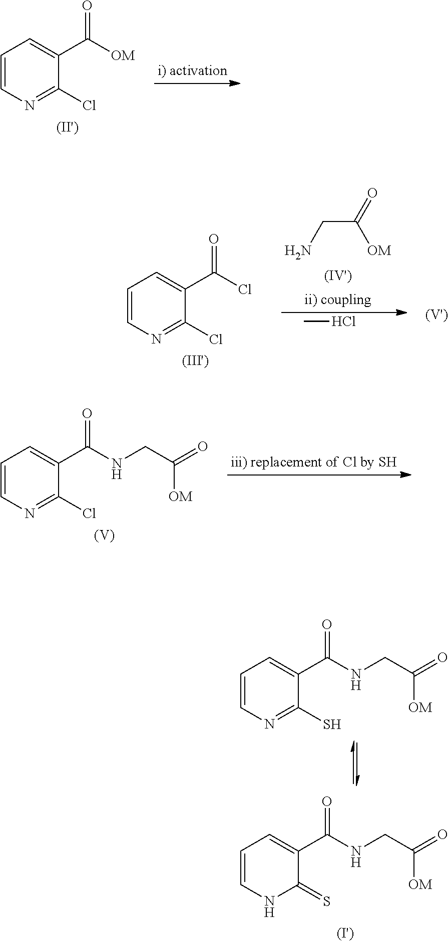

In the formulae (III′), (IV′), (V′) and (I′), M represents a hydrogen atom, an alkali metal or alkaline earth metal, such as a sodium atom or a potassium atom, or an ammonium group.

Preferably, M represents a hydrogen atom.

### Compounds (I″)

The present invention also relates to a compound of following formula (I″):

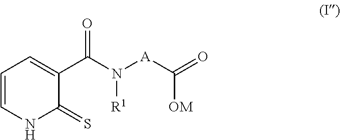

in which formula (I)
   M, A and R¹ have the same meanings as in the formula (I),
and also their optical isomers, geometrical isomers and tautomers, and also their organic or inorganic acid or base salts, and their solvates, such as hydrates,
with the exception of the following compounds (X) and (XI) and their tautomers (X′) and (XI)′:

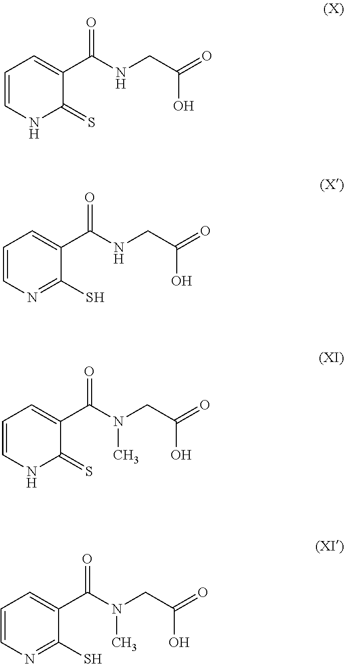

The optical isomers, the acid or base salts, the solvates and also the tautomers have the same definitions as those mentioned above.

Preferably, in formulae (I″), R¹ represents a hydrogen atom.

### Composition

A subject-matter of the invention is likewise a composition, preferably a cosmetic composition, comprising at least one compound of formula (I″) and also their optical isomers, geometrical isomers and tautomers, and also their organic or inorganic acid or base salts, and their solvates, such as hydrates.

Another subject-matter of the invention is a composition, preferably a cosmetic composition, comprising a) at least one compound of formula (I″) and also their optical isomers, geometrical isomers and tautomers, and also their organic or inorganic acid or base salts, and their solvates, such as hydrates, and b) at least one compound of formula (I) and also their optical isomers, geometrical isomers and tautomers, and also their organic or inorganic acid or base salts, and their solvates, such as hydrates, preferably at least one compound of formula (I′) and also their optical isomers, geometrical isomers and tautomers, and also their organic or inorganic acid or base salts, and their solvates, such as hydrates, it being understood that said compound of formula (I) or (I′) differs from the compounds of formula (I″) in the nature of the divalent radical A and/or in the nature of the radical R¹.

Preferably, the compound of formula (I″) can be present within the composition in a content ranging from 0.01% to 10% by weight, preferably in a content ranging from 0.1% to 5% by weight, more preferentially in a content ranging from 0.5% to 3% by weight, with respect to the total weight of the composition.

The composition according to the invention preferably comprises a physiologically acceptable medium, in particular a medium compatible with human keratin materials, such as the skin of the body or of the face, the lips, the mucous membranes, the eyelashes, the nails, the scalp and/or the hair.

The composition can additionally comprise at least one adjuvant usually employed in the cosmetic field.

Mention may be made, byway of example, of the cosmetic adjuvants chosen from the group consisting of organic solvents, in particular C₁-C₆, more preferentially C₂-C₆, alcohols and C₂-C₁₀ carboxylic acid esters; oils, in particular hydrocarbon oils and/or silicone oils, of mineral, animal and/or vegetable origin; waxes, pigments, fillers, dyes, surfactants, emulsifiers; cosmetic or dermatological active agents, UV screening agents, polymers, hydrophilic or lipophilic gelling agents, thickeners, preservatives, fragrances, bactericides, ceramides, odour absorbers and antioxidants.

These optional cosmetic adjuvants can be present in the composition in a proportion of 0.001% to 80% by weight, in particular of 0.1% to 40% by weight, with respect to the total weight of the composition.

The invention is illustrated in more detail in the following non-limiting examples.

### EXAMPLE

### Stage 1: Preparation of the Acyl Halide (Acyl Chloride)

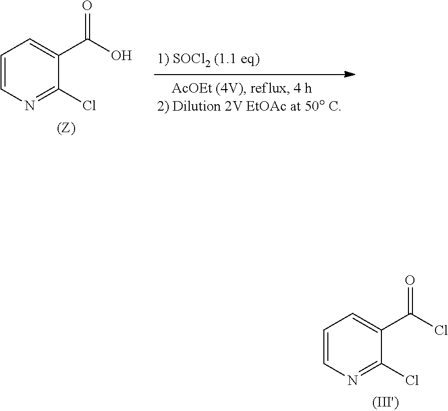 [SMILES: O=C(O)c1cccnc1Cl ; O=C(Cl)c1cccnc1Cl]

2-Chloronicotinic acid compound (Z) (70 g, 0.444 mol) and ethyl acetate (280 ml, 4.0 v/w) are introduced into a 1-litre reactor equipped with a thermometer, a condensation column and a mechanical stirrer.

The resulting white suspension is heated to solvent reflux under an inert atmosphere (nitrogen). Thionyl chloride (35.4 ml, 0.489 mol, 1.1 eq.) is added dropwise. The mixture is maintained at reflux under mechanical stirring for an additional period of time of 4 hours.

Once the reaction is complete, the mixture is cooled to a temperature of 50° C. Ethyl acetate (140 ml, 2.0 v/w) is then added and then cooling is carried out to a temperature of 25° C. and the mixture is left waiting under an inert atmosphere (nitrogen).

### Stage 2: Amide Formation

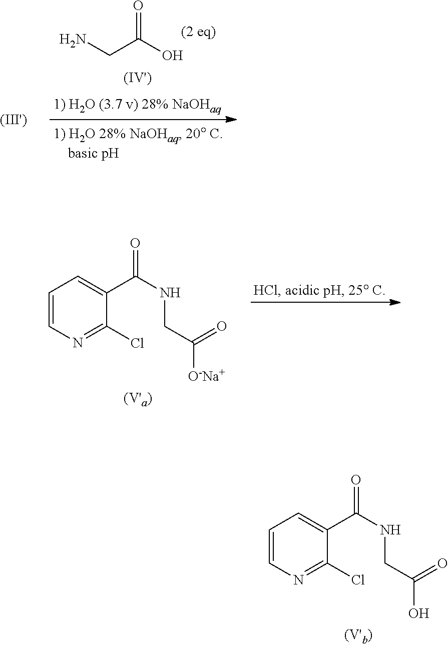

Glycine (IV′) (66.7 g, 0.890 mol, 2.0 eq.) and water (259 ml, 3.7 v/w) are introduced into a 1-litre reactor equipped with a thermometer, a pH probe and a mechanical stirrer.

The suspension is cooled to a temperature of 20±5° C. under an inert atmosphere (nitrogen) and 25% aqueous sodium hydroxide NaOH_{aq} (~56 ml. 1.0 eq.) is added until a basic pH (>7) is obtained.

A solution of acyl chloride (III′) in ethyl acetate and a 25% NaOH_{aq} solution (~84 ml, 1.5 eq.) are introduced into two separate dropping funnels. The acyl chloride solution is added dropwise to the reactor at a temperature of 20±5° C. During the addition, the pH is kept basic, preferably between 7.5 and 10, by addition of the 25% NaOH_{aq} solution.

Once the addition is complete, the solution is stirred at a temperature of 20±5° C. for a further period of time of one hour. The mixture is left to separate by settling for a period of time of 30 minutes and the aqueous phase containing the compound (V′ₐ) is collected. The ethyl acetate phase is discarded.

The aqueous phase is acidified at a temperature of 25±5° C. with an inorganic or organic acid, preferably an inorganic acid, such as hydrochloric acid, preferably 37% HCl (~58.4 ml, 1.6 eq.), down to a pH of 2.5±0.2. The suspension obtained is left stirring at a temperature of 25° C.±5° C. for approximately 1 hour.

The medium is subsequently filtered and the solid obtained is washed with water (140 ml, 2.0 v/w) and then dried at a temperature of 50° C. overnight under vacuum to result in the compound (V′_{b}) with a yield of 89% (85.3 g, white powder).

### Stage 3: Thionation

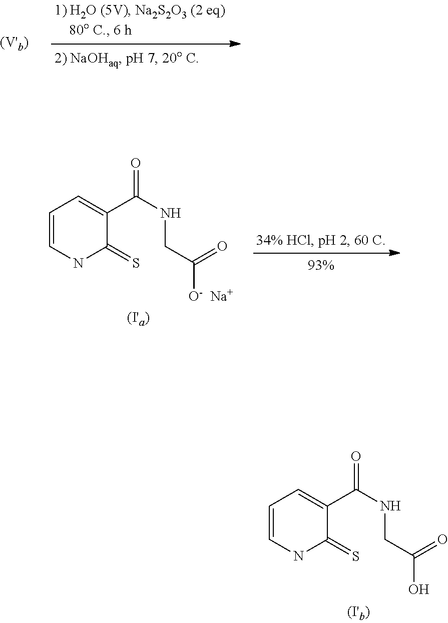 [SMILES: O=C([O-])CNC(=O)c1ccc[nH]c1=S ; [Na+] ; O=C(O)CNC(=O)c1ccc[nH]c1=S]

The compound of formula (V′_{b}) (80 g, 0.373 mol), sodium thiosulfate pentahydrate (185 g, 0.746 mol, 2.0 eq. with respect to (V′_{b})) and water (400 ml, 5 v/w) are introduced into a 1-litre reactor equipped with a thermometer, a condensation column, a pH probe and a mechanical stirrer. The resulting white suspension is heated under an inert atmosphere (nitrogen) to a temperature of 80° C.±3° C. and maintained under stirring for several hours (for example between 3 and 6 hours).

The reaction mixture is subsequently cooled to ambient temperature (20° C.±5° C.).

It should be noted that the thionation reaction is carried out at an acidic pH preferably of between 2 and 6, such as 5.

### Isolation:

At the end of the reaction, the pH of the reaction mixture is adjusted to a pH of between 7 and 8 using a 50% NaOH_{aq} solution (~35.8 g, 0.447 mol, 1.20 eq.). The mixture is left stirring at ambient temperature (20° C.±5° C.) until the pH has stabilized (for example 5 minutes). The mixture is filtered to remove elemental sulfur. The solid is rinsed with water (80 ml, 1 v/w) and the combined filtrates containing the compound of formula (I′ₐ) are transferred into an empty 1-litre reactor equipped with a thermometer, a condensation column, a pH probe and a mechanical stirrer. The solution is heated to a temperature of 60° C.±5° C. and acidified by addition of an organic or inorganic acid, preferably an inorganic acid, such as hydrochloric acid, in particular 34% HCl, until an acidic pH (for example 2.0±0.5) is obtained. The suspension is maintained under stirring at a temperature of 60° C. for a period of time of 15 minutes, before being cooled and maintained at a temperature of 20° C.±5° C. and left stirring for an additional 15 minutes. The solid is filtered off and washed with water (2×80 ml, 2×1 v/w) and then dried at a temperature of 50° C. under vacuum. The compound of formula (I′_{b}) is obtained with a yield of 93% (73.4 g, HPLC purity 99.5%) in the form of a yellow solid.

### Stage 4: Purification (Optional)

The compound prepared during the preceding Stage 3: compound (I′_{b}) (70 g, 0.330 mol) and water (280 ml, 4 v/w) are introduced into a 1-litre reactor equipped with a thermometer, a condensation column, a pH probe and a mechanical stirrer.

The pH of the mixture is adjusted to a pH of between 7 and 8 using 50% NaOH_{aq} (~29.0 g, 0.363 mol, 1.10 eq.).

The mixture is left stirring at ambient temperature (20±5° C.) until the pH has stabilized (for example 5 minutes).

The mixture is subsequently filtered in order to remove any residual elemental sulfur. The filter is rinsed with water (70 ml, 1 v/w). The filtrate is transferred into an empty 1-litre reactor equipped with a thermometer, a pH probe and a mechanical stirrer. The solution is heated to a temperature of greater than 60° C. and acidified by addition of an inorganic or organic acid, preferably an inorganic acid, such as 34% HCl, down to, for example, a pH of 2.0±0.5 (~42.5 g, 0.396 mol, 1.20 eq.).

The suspension is left stirring at a temperature of greater than 40° C., for example of greater than or equal to 60° C., for several minutes, for example 15 minutes. The suspension is cooled to ambient temperature (such as 20° C.±5° C.) and left stirring for several additional minutes (such as 15 minutes). The solid obtained is filtered off and washed with water (2×70 ml, 2×1 v/w).

The solid obtained was dried at 50° C. under vacuum to give the purified compound (I′_{b}) with a yield of 98% (68.30 g, HPLC purity 99.55%) in the form of a yellow solid.

The purity of the compounds (I′ₐ) and (I′_{b}) is measured on an HPLC system (Waters Alliance 2695 type or equivalent) equipped with a column oven and a dual wavelength UV detector or a photodiode array detector. Detection is carried out at 300 nm. The mobile phase is composed of a solution of ammonium acetate buffered at pH 3 and of methanol. The column used is a Gemini C18 5 μm—250×4.6 mm.

1. Process for the preparation of at least one compound of following formula (I): 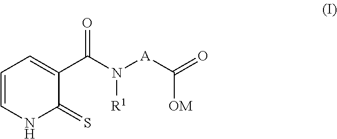
in which formula (I):
A represents a saturated or unsaturated, preferably saturated, cyclic or acyclic, preferably acyclic, linear or branched, divalent hydrocarbon chain comprising from 1 to 10 carbon atoms, optionally interrupted by one or more heteroatoms, such as an oxygen atom or a sulfur atom, or groups chosen from an NR₂ or (thio)carbonyl group, or their combinations, such as thioester, with R₂ representing a hydrogen atom or a C₁-C₄ alkyl group;
M represents a hydrogen atom, an alkali metal or alkaline earth metal, an ammonium group;
R¹ represents a hydrogen atom, a linear or branched C₁-C₁₀ alkyl group, optionally substituted by an OR₃, SR₃ group with R₃ corresponding to a hydrogen atom or a linear or branched C₁-C₁₀ alkyl group,
and also one of its optical isomers, geometrical isomers and tautomers, and also one of its organic or inorganic acid or base salts, and one of its solvates, such as hydrates;
said process taking place according to the following synthesis scheme (1): 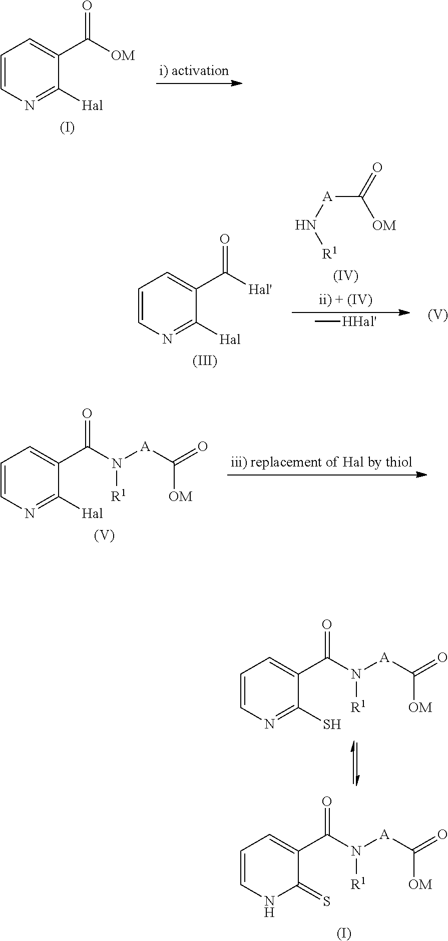
in which:
Hal and Hal′, which are identical or different, represent a halogen atom, and
M, A and R¹ have the same meanings as in the formula (I);
at least stage (iii) is at acidic pH, i.e. less than 7.

2. Process according to claim 1, characterized in that stage (iii) takes place at a pH of less than or equal to 6, more preferentially of less than or equal to 5, more preferentially still of less than or equal to 4, in particular in a pH range extending from 1 to 4, preferably from 2 to 3.

3. Process according to claim 1, characterized in that stage (iii) takes place at a pH of between 6 and 2, more particularly between 5 and 3.

4. Process according to claim 1, characterized in that stage (ii) is carried out by bringing a composition comprising at least the compound of formula (III) and at least one organic solvent into contact with an aqueous composition comprising at least the amine of formula (IV), in the presence of one or more alkaline agents.

5. Process according to claim 1, characterized in that stage (ii) comprises the addition of a composition comprising at least the compound of formula (III) and at least one organic solvent to a composition comprising at least the amine of formula (IV) and at least one alkaline agent.

6. Process according to claim 1, characterized in that stage (ii) comprises the simultaneous addition of a composition comprising at least the compound of formula (III) and at least one organic solvent and of an aqueous composition comprising one or more alkaline agents to an aqueous composition comprising at least the amine of formula (IV).

7. Process according to claim 1, characterized in that stage (ii) is carried out in a reaction medium, the pH of which is strictly greater than 7, preferably the pH of which varies from 7.5 to 10.

8. Process according to claim 1, characterized in that stage (ii) is carried out with:
at least one equivalent of compound (IV), preferably more than one equivalent, in particular between 1.5 equivalents and 2.5 equivalents, more particularly 2 equivalents, of compound (IV), preferably in a polar (a)protic solvent, preferably a polar protic solvent, such as water, in particular in a volume weight ratio of between 1 and 7, better still between 2 and 5, such as 3.5, with respect to the weight of the compound of formula (II); preferably, the reaction mixture is left at a temperature of between 10° C. and 30° C., such as 20° C.+/−5° C.; preferably, the reaction medium is left under an inert atmosphere; there is subsequently in particular added at least one inorganic or organic alkaline agent, preferably an inorganic alkaline agent, such as sodium hydroxide; at least one equivalent of alkaline agent is added to the reaction medium in order to arrive at a basic pH of said medium,
at least one compound of formula (III), in particular in a non-polar aprotic organic solvent, in particular ethyl acetate or toluene, with an alkaline agent, it being possible for said alkaline agent preferably to be in a polar (a)protic solvent, such as water, more particularly an inorganic alkaline agent, such as sodium hydroxide, preferably between 1 and 3 equivalents of alkaline agent, such as 1.5 equivalents,
preferably, the compound of formula (III) is added dropwise onto the compound of formula (IV) at a temperature of between 10° C. and 30° C., such as 20° C.+/−5° C., and preferably under an inert atmosphere and under mechanical stirring; in particular, the reaction is maintained between 1 minute and 6 hours, more particularly between 30 minutes and 2 hours, such as one hour;
more preferentially, during the addition of the compound of formula (III), said reaction medium is maintained at a basic pH, more particularly of between 7.5 and 10;
preferably, the mixture is left to separate by settling, particularly at a temperature of between 10° C. and 30° C., such as 20° C.+/−5° C., and the aqueous phase is collected;
in particular, the aqueous phase is cooled to a temperature of less than 30° C., preferably of between 20° C. and 30° C., such as 25° C.+/−5° C.; preferably, said phase is acidified with an inorganic or organic acid, preferably an inorganic acid, such as hydrochloric acid, in particular with at least 1 equivalent of acid, preferably between 1 and 3 equivalents, such as 1.6 equivalents; the pH of said aqueous phase is preferably of between 1 and 5, in particular between 2 and 4, such as 2.5+/−0.2;
preferably, the aqueous phase is subsequently filtered and the solid obtained is preferably washed with a polar (a)protic solvent, preferably a polar protic solvent, such as water; said solid is then optionally dried, preferably at a temperature of greater than or equal to 20° C., more preferentially between 30° C. and 60° C., such as 50° C.+/−5° C., to result in the compound of formula (V);
the drying stage is optional; the wet product can be used directly in the following stage.

9. Process according to claim 2, characterized in that the alkaline agent(s) can be organic or inorganic alkaline agents, preferably chosen from the group consisting of alkali metal or alkaline earth metal hydroxides, in particular sodium hydroxide, alkali metal or alkaline earth metal (bi)carbonates, in particular sodium or potassium (bi)carbonates, and tri(C₁-C₆)(hydroxy)alkylamines, in particular tri(C₁-C₆)alkylamines, especially triethylamine.

10. Process according to claim 1, characterized in that, in stage (iii), the thionation reaction of the compound (V) is carried out in the presence of a thionation agent, preferably with alkaline or alkaline earth agent thiosulfate, such as sodium thiosulfate, preferably in the presence of a polar (a)protic solvent, preferably a polar protic solvent, such as water;
preferably, the reaction medium in stage iii) is maintained under an inert atmosphere, in particular at a temperature of less than or equal to the solvent reflux, preferably at a temperature of between 50° C. and 110° C., more particularly between 60° C. and 100° C., preferentially between 70° C. and 90° C., such as 80° C.+/−3° C.; preferably for between 30 minutes and 24 hours, more preferentially between 1 hour and 10 hours, better still between 4 and 8 hours, such as 6 hours; preferably, the reaction mixture is cooled to a temperature of between 10° C. and 30° C., such as 20° C.+/−5° C.

11. Process according to claim 10, characterized in that, after the thionation stage (iii):
iii1) the reaction mixture is adjusted preferably to a pH of between 7 and 8, in particular using at least one alkaline agent as defined above, preferably an inorganic alkaline agent, such as sodium hydroxide; preferentially, the reaction mixture is subsequently left under mechanical stirring at a temperature of between 10° C. and 30° C., such as 20° C.+/−5° C., in particular for one minute to 1 hour, such as 15 minutes; preferably, the reaction mixture is filtered and the solid is washed with in particular a polar (a)protic solvent, preferably a polar protic solvent, such as water;
iii2) the filtrate is subsequently acidified, preferably at a temperature of greater than or equal to 40° C., such as 60° C.+/−5° C., using an organic or inorganic acid, preferably an inorganic acid, such as hydrochloric acid, until a pH of between 1 and 5, preferably between 1.5 and 3, such as 2+/−0.5, is obtained; the temperature is subsequently preferably maintained at 60° C.+/−5° C. for a period of time of in particular between 1 minute and 30 minutes, such as 15 minutes;
preferentially, the reaction mixture is subsequently left under mechanical stirring at a temperature of between 10° C. and 30° C., such as 20° C.+/−5° C., in particular for one minute to 1 hour, such as 15 minutes; preferably, the reaction mixture is filtered and the solid is washed with in particular a polar (a)protic solvent, preferably a polar protic solvent, such as water; preferably, the solid is dried at a temperature of between 20° C. and 60° C., such as 50° C., in particular under vacuum;
iii3) the solid can be purified by dissolving it in a polar (a)protic solvent, preferably a polar protic solvent, such as water, by adjusting the pH between 7 and 8 using in particular an alkaline agent, preferably an inorganic alkaline agent, such as sodium hydroxide; the mixture is subsequently preferably maintained under mechanical stirring, in particular between 10° C. and 30° C., such as 20° C.+/−5° C., for between 1 minute and 30 minutes, such as 5 minutes; the mixture is subsequently preferably filtered and the solid washed with a polar (a)protic solvent, preferably a polar protic solvent, such as water;
iii4) the filtrate is subsequently optionally acidified, depending on the nature of M, using an inorganic or organic acid, preferably an inorganic acid, such as hydrochloric acid, in particular at a temperature of greater than or equal to 40° C., such as 60° C.+/−5° C.; preferably, the pH is maintained between 1 and 4, more preferentially between 1.5 and 3, such as 2+/−0.5; preferably, the mixture is subsequently left at a temperature of greater than or equal to 40° C., such as 60° C.+/−5° C., for in particular between 1 and 60 minutes, such as 15 minutes; the solid is subsequently filtered off and washed with a polar (a)protic solvent, preferably a polar protic solvent, such as water, followed in particular by drying at a temperature of greater than or equal to 20° C., more particularly at a temperature of between 40° C. and 60° C., such as 50° C., preferably under vacuum.

12. Process according to claim 4, characterized in that the ratio by volume of the organic solvent to the water can vary from 3 to 1, preferably is equal to 2/1.

13. Process according to claim 1, characterized in that:
A represents a (C₁-C₆)alkylene group, more preferentially a (C₁-C₄)alkylene group, in particular methylene; and/or
R¹ represents a hydrogen atom.

14. Process according to claim 1, characterized in that the compound(s) of formula (I) is or are chosen from the group consisting of the compounds corresponding to the following formula (I′): 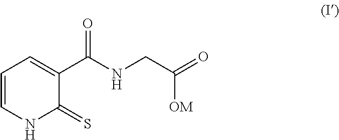
in which formula (I′) M represents a hydrogen atom, an alkali metal or alkaline earth metal, such as a sodium atom or a potassium atom, or an ammonium group; preferably, M represents a hydrogen atom.

15. Compound of following formula (I″): 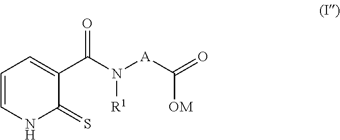
in which formula (I″):
A represents a saturated or unsaturated, preferably saturated, cyclic or acyclic, preferably acyclic, linear or branched, divalent hydrocarbon chain comprising from 1 to 10 carbon atoms, optionally interrupted by one or more heteroatoms, such as an oxygen atom or a sulfur atom, or groups chosen from an NR₂ or (thio)carbonyl group, or their combinations, such as thioester, with R₂ representing a hydrogen atom or a C₁-C₄ alkyl group;
M represents a hydrogen atom, an alkali metal or alkaline earth metal, an ammonium group;
R¹ represents a hydrogen atom,
and also their optical isomers, geometrical isomers and tautomers, and also their organic or inorganic acid or base salts, and their solvates, such as hydrates,
with the exception of the following compounds (X) and (XI) and their tautomers (X′) and (XI)′:

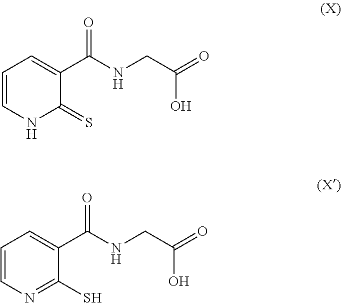 [SMILES: O=C(O)CNC(=O)c1ccc[nH]c1=S ; O=C(O)CNC(=O)c1cccnc1S]

16. Composition, preferably cosmetic composition, comprising one or more compounds of formula (I″), as described according to claim 15, and also their optical isomers, geometrical isomers and tautomers, and also their organic or inorganic acid or base salts, and their solvates, such as hydrates.
